(19) United States
(12) Patent Application Publication
(10) Pub. No.: US 2010/0129377 A1
(43) Pub. Date: May 27, 2010

(54) MILK DERIVED ANTIGEN-SPECIFIC ANTIBODIES, METHODS OF PREPARATION AND USES THEREOF

(75) Inventor: Ruprecht Jules Joost Van Neerven, Almere (NL)

Correspondence Address: MORRISON & FOERSTER LLP, 12531 HIGH BLUFF DRIVE, SUITE 100, SAN DIEGO, CA, 92130-2040, US

(73) Assignee: Friesland Brands B.V., Meppel (NL)

(21) Appl. No.: 12/596,139

(22) PCT Filed: Apr. 16, 2008
(86) PCT No.: PCT/NL2008/050214

§ 371 Date: Feb. 10, 2010

(30) Foreign Application Priority Data
Apr. 16, 2007 (EP) 07106250.9
Apr. 16, 2007 (NL) 1033696
Apr. 16, 2007 (US) 60912001

(51) Int. Cl. A61K 39/395; C07K 16/00; A61P 37/04

(52) U.S. Cl. 424/157.1; 530/387.1; 530/389.1

(57) ABSTRACT

The invention relates to means and methods for obtaining an antigen specific antibody from milk from non-human mammals that have not been immunized with said antigen prior to collecting said milk. Non-human mammal derived antigen specific antibody was found to cross the mucosal side of the intestinal tract of an individual. The invention provides novel means and methods for the treatment of disease through antigen specific non-human mammal antibodies. Further provided are preparations for oral administration comprising said antigen specific antibody of a ruminant.

The invention relates to the field of antibodies. In particular the invention relates to milk-derived antigen specific antibodies, methods of preparation and uses thereof. Such uses entail for instance, in infant nutrition, in diagnostic and medical uses.

There are several ways to enhance human immunity by providing dairy foods. Mother's milk and cow's milk contain antibodies that are specific for a wide range of potential pathogenic microorganisms, viruses and other antigens. These antibodies protect the infant that is not yet able to mount an effective immune response against infectious agents that the mother has already been exposed to. IgA is the predominant antibody in human milk, but IgG₁ is the predominant antibody isotype found in bovine milk. The IgA in human milk is generally known to exert its effect in the intestine, whilst the mother transfers IgG to the baby via the placenta before birth. Most ruminants, pigs and rodents actively transport a portion of the IgG present in milk over the intestinal epithelium into the blood. These animals thus, like humans, also have antibodies in the milk of lactating females, particularly in the first milk after birth (colostrum) albeit that the actual function of these antibodies in the neonate has additional properties as compared to antibodies in the milk of humans.

As ruminants produce milk that contains antibodies, the milk or antibody preparations thereof are considered for use in humans to supplement or replace the function of mother milk. To enable collection of antigen specific antibodies in the milk ruminant are typically immunized with the particular antigen for which antibodies are desired. The antigen is typically a human pathogen, for instance an intestinal microbe or virus and the antibodies in the milk of these immunized animals are collected. The collected antibodies are intended to bind to and neutralize or otherwise impair the human pathogen in the intestine of individuals ingesting the antibodies.

Apart from milk, colostrum is another source for isolating bovine antibodies. Bovine colostrum is the first milk produced by a cow just before and shortly after giving birth to a calf. Colostrum contains very high levels of bovine antibodies, up to 50-100 mg/ml (vs around 1 mg/ml for normal milk). IgG₁ is the predominant antibody isotype in bovine colostrum and in milk. As mentioned above, the immunoglobulins in colostrum are absorbed into the blood by calfs to protect them against infection. The antibody levels in colostrums are about 50-100 fold higher than in normal milk. For this reason colostrum of hyperimmunized cows has been used as a source of bovine antibodies to treat and prevent diarrhea in a number of studies showing significant effects on diarrhea symptoms. (Clin infect dis 1998 26:1324-9 adults; Acta pediatr. 1995 84:996-1001 infants; Acta paediatr. 2001 90 1373-8 children 1-12 y; J. Pediatr. Gastroenterol Nutr 1999 29:452-6). Some of these studies are reviewed in Indian J. Pediatr. 2005 72:849-52.

In addition to the use in preventing/treating diarrhea, colostrum is also used as a supplement by athletes (eur. J. Nutrition 2003 42:228-232 and Int. J. Sport Ntr. Excerc Metab 2006 16:47-64. J. Appl Physiol 2006 93:732-39).

In another study (JT van Dissel at al J. Med. Microbiol 2005 54-197-205) a 40% whey protein concentrate of cows immunized with Clostridium difficile was used to prevent relapses of C. difficile diarrhea—Results are preliminary, but no adverse events noted due to MucoMilk (Scand J. Gastroenterol 2000 35:711-8; N. Engl J Med 1988 318:1240-3.

These data show that colostrum from cows contains high antibody levels and that oral administration of these antibodies in adults and infants is without adverse effect, in at least these studies, indicating that the use of cow antibodies in food is safe.

Example 4 and example 5 of EP0152270B1 indicate that no serum sickness arises after ingestion of bovine milk and injection of purified IgG in humans. Rabbits drinking milk before the injection with IgG do not produce anti bovine IgG antibodies. These examples again indicate safety and oral tolerance induction to bovine IgG.

In the present invention it was found that antibodies derived from milk of ruminants pass the mucosal barrier in the human intestine and can bind to and act against antigen present on the bloodstream side thereof. Antibodies thus cross the epithelial lining of the intestine and are present at the basolateral side of the intestinal epithelial cells. Without being bound by theory it is believed that antibodies derived from the milk of a ruminant bind to an Ig-receptor on human intestinal cells and are transported from the intestinal side to the bloodstream side of the intestinal mucosa. In any case antibodies provided to the apical side of intestinal epithelial cells pass through the lining to the basolateral side. The invention thus provides a method for providing the basolateral side of intestinal epithelial cells with an antibody through administering said antibody to the apical side thereof. In a preferred embodiment the transport over the epithelial cells is further enhanced by providing the apical side thereof with a prebiotic carbohydrate. Prebiotic carbohydrates are non-digestible carbohydrates (oligo- and polysaccharides) as described in Gibson and Roberfroid et al (1995, J. Nutr. 125: 1402-1412. The prebiotic component is preferably lacto-N-tetaose, lacto-N-fuco-pentaose, lactulose (LOS), lactosucrose, raffinose, galacto-oligosaccharide (GOS), fructo-oligosaccharide (FOS), oligosaccharides derived from soybean polysaccharides, mannose-based oligosaccharides, arabin-oligosaccharides, xylo-oligosaccharides, isomalto-oligo-saccharides, glucans, sialyl oligosaccharides, and fuco-oligosaccharides. The prebiotic is further preferably an oligosaccharide produced from glucose, galactose, xylose, maltose, sucrose, fucose, arabinose, glucosamine, mannose, lactose, starch, xylan, hemicellulose, inulin, or a mixture thereof. Most ruminants, pigs, and rodents have a specific neonatal receptor to transport IgG across the intestinal epithelium. This receptor is termed FcRN, the neonatal Fc receptor that is expressed shortly after birth and is downregulated by the time of weaning. Humans constitutively express FcrN on intestinal epithelia (Immunology 92:69-74) and bidirectional FcRN-dependent transport of human IgG over epithelial cells has been shown in humans (J. Clin Invest 1999 104:903-911). FcRN is also expressed on myeloid cells in humans (J. Immunol. 2001 166:3266-76).

pIgR is the polymeric Ig receptor. This receptor binds to the j-chain that is needed to form dimeric IgA and pentameric IgM. This receptor is therefore thought to transport both IgA and IgM. A recent publication has indicated that transport of antigen-complexed IgA from the intestinal lumen into the tissues can also occur (J. Gen Virol 2005 86:2747-51). CD23, the low affinity IgE receptor is also expressed on intestinal epithelium, and has been shown to play a role in the internalization of food allergens in animal models (gastroenterology 2006 131: 47-58; J. Clin Invest. 2000: 106:879-886).

The invention therefore provides a method for delivery of an antigen specific antibody of a ruminant to the mucosal side, i.e. basolateral side of the epithelium, of the intestinal tract of an individual comprising providing a preparation for oral administration comprising said antigen specific antibody of a ruminant and orally administering said preparation to said individual. Ruminant antibody provided crosses the intestinal epithelium and is able to enter the intestinal mucosa and subsequently enter the bloodstream and can interact with the antigen it is specific for.

A ruminant antibody for use in crossing the intestinal epithelium can be derived from a ruminant that has not been previously immunized for the antigen that said antibody is specific for. Alternatively, the ruminant antibody can be derived for a ruminant that has been previously immunized with the antigen that said antibody is specific for. Although ruminant antibodies are relatively resistant to degradation in the human gastro-intestinal tract it is preferred that said preparation for oral administration comprises a delivery vehicle comprising said antigen specific antibody, wherein said delivery vehicle enables targeted intestinal release of said antibody from said vehicle. Various delivery vehicles are known in the art that allow specific release of enclosed or enveloped substances in the intestinal tract. It is preferred that said vehicles allow release in the small intestine. Preferably said preparation further comprises a buffer for maintaining and/or achieving a local pH in the intestine that is favourable for uptake and/or functionality of the antibody. Preferably said buffer is set to achieve a pH between about 6-8 in the small intestine. In a preferred embodiment said antibody is obtained from the milk of a lactating ruminant. Milk of said ruminant contains, when compared for instance with serum, a large proportion of IgG₁. IgG₁ is one of the ruminant antibody subtypes that is transported across the human intestinal mucosa. As the antibodies pass through the mucosa they can act beyond the interior of the gastro-intestinal tract. In one embodiment a ruminant antibody is specific for an allergy antigen. In this way the allergen, upon uptake by the intestine, is bound in and preferably on the bloodstream side of the intestinal mucosa (i.e the basolateral side of the epithelium) thereby at least competing for binding to said antigen with human IgE specific for said antigen. In further preferred embodiment said antigen specific antibody is specific for a food allergy-associated antigen, preferably a food allergy associated antigen from soy, hazelnut, egg or apple. In a particularly preferred embodiment said allergy antigen is an allergy-associated peanut antigen. In another embodiment said allergen is an inhalation allergen or a venom allergen selected from but not limited to the group of grass pollen allergens, tree pollen allergens, house dust mite allergens, cat allergens, mould or fungus allergens, or stinging insect venoms. Said inhalation and venom allergens can enter the human body via the respiratory tract or via a sting of an insect such as a bee or a wasp.

In another preferred embodiment said in individual is suffering from an auto-immune disease or otherwise over-active immune system. In this embodiment it is preferred that said antigen specific antibody of the ruminant is specific for a proinflammatory cytokine or a stimulatory signalling factor produced by immune cells of the individual and that contribute to the maintenance and/or severity of a symptom of the auto-immune disease or otherwise over-active immune system. Thus preferably an antibody of the invention is specific for a factor excreted by immune cells. In a particularly preferred embodiment a ruminant antibody of the invention is specific for human TNF-α or human IL-23 antibody. In yet another embodiment said antibody is specific for an immune cell, an epithelial cell, an intraepithelial cell, an antigen presenting cell or an M-cell of the individual to be treated, preferably a T-cell, a B-cell or a dendritic cell. In a preferred embodiment said individual is suffering from inflammatory bowl disease. In a further preferred embodiment said ruminant antibody of the invention is specific for an antigen of a microorganism associated with said auto-immune disease.

The human may be of any age. However, it is preferred that said human individual is at least one year old. Preferably said individual is at least two years old. Preferably said individual is at least 6 years old. In another preferred embodiment said individual is at least 16 years old. In yet another aspect of the invention said individual is between 0 and 24 months old, preferably between 0 and 12 months, more preferably between 6 and 24 months old (typically when solid food is included in the diet). The gastroinstestinal tract of individuals of between 0-6 months is not yet completely developed and more easily allows passage of the antibody to the basolateral side of the intestinal epithelial cells as compared to an adult. In addition, antibodies have a longer half life in the gastro-intestinal tract of individuals of between 0-24 months old. In a preferred embodiment said antibody is of type IgG₁, IgA or a combination thereof. Preferably, said antibody is of the IgG₁ type or composition comprising both IgG₁ and IgA specific for said antigen. A method of the invention is preferably used to deliver said ruminant antibody to the bloodstream via the gastrointestinal tract of said individual. In a particularly preferred embodiment said bloodstream delivery is used to deliver the antibody to the lung. In this embodiment it is preferred that the antibody is specific for an antigen of a viral or a bacterial pathogen or an antigen of an allergy. In this embodiment it is preferred that said antibody is specific for a allergy-associated grass- or tree-pollen antigen, or any other inhalation allergen that causes allergic reactions in humans. In another preferred embodiment said antibody is specific for a micro-organism or a virus with a tropism for the lung. Preferably said micro-organism comprises Streptococcus pneumoniae, Staphylococcus aureus, Haemophilus influenzae, Escherichia coli, Pseudomonas aeruginosa, Moraxella catarrhalis, Chlamydia pneumoniae, Mycoplasma pneumoniae and Legionella pneumophila. Preferred virus specific antibodies are directed against a member of the influenza viruses, preferably influenza virus A or influenza virus B, a member of the respiratory synctial viruses or a member of the adenoviruses.

The invention thus provides the use of an antigen specific antibody of a ruminant for the preparation of a medicament for systemic treatment of a disease in a non-ruminant. Preferably said non-ruminant is a human. Preferably said medicament is for oral administration.

A particular advantage of an antibody of the invention is that it is a polyclonal antibody. This allows for more robust antigen binding. This further allows for reduced variation between individuals provided with an antibody of the invention.

In a further aspect of the invention it was found that milk from a non-human mammal that was not previously immunized for an antigen can specifically react with said antigen. This indicated to us that the milk contained antibodies that specifically bound the antigen in spite of the fact that the non-human mammal had not been immunised for said antigen. In the present invention this aspect is exploited in an industrial setting were large volumes of milk are processed. In this aspect the invention thus provides a method for obtaining an antigen specific antibody from milk characterized in that the milk is derived from a mammal that has not been immunized with said antigen prior to collecting said milk. With immunization for an antigen is meant administering a composition comprising the antigen and typically an adjuvant to a mammal with the intention of obtaining an immune response against said antigen, said adjuvant or combination thereof in said mammal. The administration involves the facilitation of intensive contact between the components of the composition and the immune cells of the mammal. Such administrations typically involve depositing the composition inside the mammal by breaking the skin layer. Other means of administration exist and can be used. Oral administration of antigen as, for instance (part of) the food of said mammal, is often associated with the induction of tolerance in the mammal for said antigen and is therefore not considered an immunization in the present invention. In a preferred embodiment said method comprises collecting milk from a lactating mammal and collecting said antibody from said milk. Preferably milk of at least two individual mammals of the same species is pooled prior to collecting said antibody. In this way, also low abundant antibodies can be collected in sufficient amounts. For this purpose it is preferred to collect antibodies from milk of at least 10 and more preferably at least 100 individual mammals preferably of the same species. In a preferred embodiment said mammal is a ruminant. Preferably said ruminant is cow, preferably a member of the genus Bos. Of the genus Bos, the commercially exploited species are preferred. Preferably said ruminant is of the species Gayal, Bos frontalis (domestic gaur), Bos mutus (Yak) or Bos taurus (Domestic Cattle). In another preferred embodiment said ruminant is a goat or a sheep, preferably a member of the subfamily Caprinae. Of this subfamily the members of the genera Ovis or Capra are preferred. Of the genus Ovis, the species Ovis aries (Domestic Sheep) is preferred. Of the genus Capra the species Capra aegagrus hircus (the domesticated goat) is preferred. In another preferred embodiment said ruminant is a camel, a donkey, a buffalo, a horse or a lama. Milk from non-immunized ruminants contains antibodies specific for a variety of different antigens. Preferred preparations contain antibodies directed toward a member of the genus Escherichia, preferably Escherichia coli, a member of the genus Salmonella, preferably Salmonella typhimurium, a member of the genus Campylobacter, preferably Campylobacter jejuni, a member of the genus Helicobacter, preferably Helicobacter pilori, a member of the genus Bordotella, preferably Bordotella pertussis, a member of the genus Clostridium, a member of the genus Shigella, a member of the genus Streptococcus, preferably Streptococcus pneumoniae, a member of the genus Staphylococcus, preferably Staphylococcus aureus, a member of the genus Haemophilus, preferably Haemophilus influenzae, a member of the genus Pseudomonas, preferably Pseudomonas aeruginosa, a member of the genus Moraxella, preferably Moraxella catarrhalis, a member of the genus Chlamydia, preferably Chlamydia pneumoniae, a member of the genus Mycoplasma, preferably Mycoplasma pneumoniae or a member of the genus Legionella, preferably Legionella pneumophila. Preferred virus specific antibodies are directed against a member of the genus influenza, preferably influenza virus A or influenza virus B, a member of the respiratory synctial virus, a member of the rotaviruses, a member of the novoviruses, a member of the enteroviruses, a member of the cytomegaloviruses or a member of the adenoviruses.

As milk contains several components, notably fats, oils non-antibody protein and carbohydrates it is preferred to submit said milk to a processing step prior to collecting said antibody. A suitable starting preparation for the collection of antibodies is raw milk. Milk that is treated with heat to achieve a logarithmic kill of bacteria, a process referred to as pasteurization, can also be used. Pasteurisation typically uses temperatures below boiling since at temperatures above the boiling point for milk, casein micelles will irreversibly aggregate (or “curdle”). There are two main types of pasteurization used today: High Temperature/Short Time (HTST) and Extended Shelf Life (ESL) treatment. In the HTST process, milk is forced between metal plates or through pipes heated on the outside by hot water, and is heated to 71.7° C. (161° F.) for 15-20 seconds. ESL milk has a microbial filtration step and lower temperatures than HTST. Milk simply labeled “pasteurized” is usually treated with the HTST method. Milk can also be pasteurized at temperatures lower than 71.7° C. in combination with increased pressure. This is equally effective in logarithmic killing of bacteria as higher temperatures at lower pressure.

A milk processing step preferably comprises a separation step. A preferred separation step comprises a step wherein the milk is (partially) depleted for fat. Thus another preferred starting preparation for the collection of antibodies comprises milk that has undergone a fat depletion step. In a preferred embodiment said separation step comprises separating the milk in at least two parts and of which at least one is a protein rich part. Collection of antibody from batch processed milk is possible, however, it is preferred that said processing step is part of a (semi)-continuous process. In a particularly preferred embodiment a whey fraction is prepared from said milk and said antibody is collected from said whey. Thus in a preferred embodiment the starting preparation for the collection of antibodies comprises whey. In yet another embodiment the starting preparation for the collection of antibodies comprises fermented milk. A starting point can be as indicated herein above but also from a reconstituted concentrate or dry powder produced therefrom. The mammal is as mentioned selected on the criterion that it has not previously been immunized for said antigen. In a preferred embodiment said mammal is selected on a further criterion for collection of said milk. This further criterion can be any criterion, for instance, time in the lactation cycle. In a preferred embodiment, said mammal is selected on the basis that it is producing normal milk, i.e. not the milk, also referred to as colostrum, immediately after giving birth. In further preferred embodiment said further criterion comprises antibody content of the milk, antibody specificity in collected milk, type of food or type of food-supplement ingestion by said mammal, vaccination for an antigen of a pathogen of said mammal or a combination of two or more of said criteria.

In a preferred embodiment said antibody is an IgG₁, IgA, IgG₂ or an IgM antibody. Preferably said antibody is collected from an antibody-enriched fraction obtained from said milk. Preferably said method further comprises a step to enhance the levels of IgG₁, IgA or a combination thereof when compared to other proteins present in the antibody enriched fraction. Preferably the amount of β-lactoglobulin is decreased by at least 50%. This can be accomplished for example by a specific hydrolysis of β-lactoglobulin, or by removal of β-lactoglobulin by size separation or by removal using an antibody specific for β-lactoglobulin. Thus preferably, a method for collecting an antibody of the invention further comprises a step to reduce the amount of β-lactoglobulin in the antibody enriched fraction. A method of the invention preferably further comprises an affinity purification step to obtain an antibody fraction that is enriched for said antigen specific antibody. Having the possibility to collect antigen specific antibody from a large collection of different mammals of the same species allows the affinity purification of significant amounts of affinity purified antigen specific antibody, in spite of a possible low prevalence of said antibody in each individual milk sample.

Further provided is an antigen specific antibody obtainable by a method for collecting an antigen specific antibody according to the invention. Preferably said antibody is a polyclonal antibody. In this embodiment said antigen specific antibody can be derived from an immunized non-human mammal or from a non-immunized non-human mammal. In a further preferred embodiment said antigen is an allergy antigen, a viral antigen, a bacterial antigen or a combination thereof.

The term antigen in the context of the present invention is an antigen of a human, a pathogen able to infect and cause disease in a human, an allergen to which a human can become allergic or a combination thereof. Immunization of a mammal in the context of the present invention thus means immunization of said mammal with an antigen of a human, a pathogen able to infect and cause disease in a human, an allergen to which a human can become allergic or a combination thereof. Vaccination of said mammal for an antigen of a pathogen of said mammal to treat or prevent a disease in said mammal is therefore not considered an immunization in the context of the present invention.

The invention further provides a composition comprising an antigen specific antibody of a non-human mammal of the invention. Preferably said antigen specific antibody comprises less than 1% of the total amount of antibody in said composition. More preferably said antigen specific antibody comprises less than 0.1% of the total amount of antibody in said composition. In a preferred embodiment at least 15% and more preferably at least 25% and more preferably at least 35% and more preferably at least 50% and more preferably at least 60% and more preferably at least 75% even more preferably at least 80% of the antibodies are IgG₁, IgA or a combination thereof. Preferably said composition is enriched for IgG₁ over IgA. In another preferred embodiment of the invention the composition is enriched for IgA over IgG₁.

The invention further provides a composition comprising antibody of a non-human mammal of the invention wherein said composition comprises antibody in amount that is between 0.001 to 30% of the total amount of protein in said composition. Preferably said antibody is present in amount of 0.1 to 30% % of the total amount of protein in said composition. Preferably said antibodies are present in an amount of between about 5-30%, more preferably between 10-30%, more preferably between about 20-30% of the total amount of protein. In a preferred embodiment at least 10%, more preferably at 15% and more preferably at least 25% and more preferably at least 35% and more preferably at least 50% and more preferably at least 60% and more preferably at least 75% even more preferably at least 80% of the antibodies are IgG₁, IgA or a combination thereof. Preferably said composition is enriched for IgG₁ over IgA. In another preferred embodiment of the invention the composition is enriched for IgA over IgG₁.

The invention further provides a pharmaceutical composition comprising an antigen specific antibody of a non-human mammal according to the invention, or a composition according to the invention. Yet further provided is the use of an antigen specific antibody of a non-human mammal according to the invention, or a composition according to the invention, for the preparation of a medicament. In a preferred embodiment is provided the use of an antigen specific antibody of a non-human mammal according to the invention, or a composition according to the invention for the preparation of a medicament for oral administration.

In yet a further aspect the invention provides a preparation for oral administration comprising an antigen specific antibody of a non-human mammal according to the invention, or a composition according to the invention. In a preferred embodiment said preparation comprises a delivery vehicle comprising said antibody or composition. Said delivery vehicle preferably enables targeted intestinal release of said antibody from said vehicle.

In yet another aspect the invention provides a newborn or infant formula comprising a preparation according to the invention as mentioned herein above. Also provided is a newborn or infant formula comprising an antigen specific antibody of a non-human mammal according to the invention, or a composition according to the invention. Also provided is a newborn or infant formula, wherein between 0.001 and 5% of the protein content of said formula is antigen specific antibody protein of a non-human mammal according to the invention, preferably said formula comprises at least 0.1, more preferably at least 1, more preferably at least 2, more preferably at least 5% of the protein content of said formula is antigen specific antibody protein of a non-human mammal according to the invention. Preferably antibodies of non-human mammals of the invention are present in an amount of between about 1-30%, more preferably between 10-30%, more preferably between about 20-30% of the total amount of protein. Preferably at least 5, more preferably at least 10% and more preferably at least 20% of the protein content of said formula is antibody protein of the non-human mammal. Preferably between about 5-10% of the protein content of said formula is antibody protein. More preferably between about 10-30% of the protein content of said formula is antibody protein.

In yet another embodiment is provided a newborn or infant formula is indicated herein above, wherein at least 0.001, more preferably at least 0.01, more preferably at least 0.1, more preferably at least 1, more preferably at least 2, more preferably at least 5% of the protein content of said formula is antigen specific antibody protein. Preferably between about 0.01-5% of the protein content of said formula is antigen specific antibody protein of a non-human mammal according to the invention. Thus preferably between about 0.001-5% of the protein content of said formula consists of an antigen specific antibody according to the invention, or a composition according to the invention.

The invention further provides a food supplement or a food for a non-human animal or human comprising an antibody of the invention and/or a composition of the invention. In this embodiment said antigen specific antibody can be derived from an immunized non-human mammal or from a non-immunized non-human mammal.

An antibody from an immunized or non-immunized non-human mammal is preferably specific for an allergen antigen, a human growth factor, a human antigen, a respiratory virus, respiratory bacterium, a parasite, or other relevant diseases-related antigen. In a preferred embodiment said antibody is derived from an immunized non-human mammal.

A bovine and/or non-human mammal antibody of the invention can be administered orally as a food product or nutraceutical, a drink, a capsule or an encapsulated antibody-containing preparation. A bovine and/or non-human mammal antibody from a non-immunized or from an immunized bovine and/or non-human mammal can also be administered via other routes such as via intramuscular injection, intravenous administration, or via the rectum or vagina.

An orally delivered bovine and/or non-human mammal antibody preparation can be formulated in a fluid such as a watery solution, milk, yoghurt, or as a freeze dried protein preparation, or encapsulated to protect the antibody in the stomach. Preferably, such antibody is added to an infant formula or a milk product meant for human consumption. In another preferred embodiment of the invention, said antibody is encapsulated, combined with a carrier, with a probiotic bacteria, or with a prebiotic (e.g. galactooligosaccharides).

Further provided is a nutraceutical containing an antigen specific antibody of a non-human mammal according to the invention, or a composition according to the invention. Said nutraceutical take any form that is suitable for oral consumption. Preferably said nutraceutical is a capsule, encapsulated protein, or liquid solution. The nutraceutical is intended to prevent, treat, or at least in part alleviate symptoms of an ongoing disease. Preferably said disease is food allergy (food allergen-specific antibodies; peanut, soy, hazelnut, egg, apple, etc), inflammatory bowel disease (TNF-α or IL-23 specific antibodies), pollen allergy (grass- and tree pollen-specific, preferably birch pollen-specific antibodies), other inhalation allergies (housedust mite, cat, fungi etc), or venom allergies. Thus in a preferred embodiment the antibody preparation prepared using a method of the invention comprises an antibody specific for a food allergen antigen; preferably a peanut, soy, hazelnut, egg, apple, etc allergy antigen), or specific for an inflammatory bowel disease antigen (preferably TNF-α or IL-23 specific antibodies), or specific for a pollen allergy antigen (grass- and tree pollen-specific, preferably birch pollen-specific antibodies), or specific for other inhalation allergy antigens (housedust mite, cat, fungi etc), or specific for venom allergy antigens.

### BRIEF DESCRIPTION OF THE DRAWINGS

FIG. 1: Binding of bovine IgG1 (left panels) and IgA (right panels) antibodies from colostrum, milk, and whey to serially diluted LPS from Salmonella typhymurium (Top panels) and Escherichia coli (bottom panels). Values are expressed as OD measured at a wavelength of 450 nm.

FIG. 2: Binding of bovine IgG1 and IgA antibodies from colostrum to serially diluted allergens (house dust mite, birch pollen, and grass pollen allergens). Values are expressed as OD measured at a wavelength of 450 nm.

FIG. 3: Transepithelial transport of IgG1 and IgA in colostrum (top panels) and the effect of the transport experiment across a pH gradient on TEER and FD4 transport. Error bars indicate standard deviations of the ELISA measurements. The horizontal line in the top panels indicates the amounts of IgG1 and IgA measured in control cultures to which no colostrum was added.

### EXAMPLES

### Example 1

### Effect of Milk-Derived Antibodies from Non-Immunized Cows on Adhesion of Bacteria to Human Epithelium

The human small intestinal epithelial cell line Caco-2 as well as the human mucin producing colon epithelial cell line HT29-MTX is cultivated in Transwell plates and allowed to differentiate into monolayers during a 15-21 day culture period. Monolayer integrity is measured every week by measuring the transepithelial electrical resistance (TEER) before use in adhesion assays. Radiolabeled bacteria (two pathogenic bacteria: enteropathogenic Escherichia coli and Salmonella typhimurium, as well as Lactobacillus.casei as a probiotic strain) are incubated with the epithelial cells at a pre-determined range of microorganisms to epithelial cells (e.g. 10:1, 100:1 and 500:1) for a set period of time and the unbound microorganisms are washed off. Adherent bacteria are quantified by d.p.m. (disintegrations per min.) counting. The experiment is performed in the presence or absence of a bovine antibody preparation at (corresponding to 464 ug/ml and 46.4 ug/ml IgG1). The adhesion of the bacteria to Caco-2 and HT29-MTX is reduced by the antibody preparation.

### Example 2

### Pathogen-Specific Antibody Levels Present in Milk-Derived Antibody Preparations

Sandwich ELISAs to measure E. coli-specific, Salmonella-specific, and Clostridium-specific antibodies are used to quantify the amounts of bacteria-specific IgG1, IgM, and IgA in a preparation containing bovine antibodies. 96 well ELISA plates are coated with 100 μL heat killed bacteria (E. coli, Salmonella, Clostridium) in PBS. The plates are incubated overnight at 4° C. in a humidified environment. The plates are washed three times with 200 ul wash buffer per well (PBS+0.05% Tween-20) and blocked for 1 hour at 37° C. in a humidified environment with 200 μL block buffer per well (=wash buffer+1% gelatin). After this incubation, the plates are washed three times with 200 ul wash buffer per well (PBS+0.05% Tween-20), and 100 μL/well of bovine antibody containing sample diluted in block buffer is added.

After a 1 hour incubation at 37° C. in a humidified environment, the plates are washed three times with 200 ul wash buffer per well, and 100 μL anti-bovine IgG1-HRP (AbD Serotec, cat# AAI21P) diluted 1:50.000 in block buffer is added per well to detect bacteria-specific IgG1. For detection of IgA 100 μL anti-bovine IgA-HRP (AbD Serotec, cat# AAI20P) diluted to 1:50.000 in block buffer, and for detection of bovine IgM 100 μL anti-bovine IgM-HRP diluted to 1:50.000 in block buffer is used (AbD Serotec, cat# AAI19P).

The plates are incubated for 1 hour at 37° C. in a humidified environment, washed three times with 200 ul wash buffer per well, followed by a fourth wash with PBS, and 100 μL TMB substrate is added to each of the wells (100 μL TMB in 10 mL substrate buffer both from Biosource Int. Cat # 45.011.03 and 45.014.01).

The color reaction is stopped by the addition of 100 μL/well of 1M H2SO4, and the plates are read at a wavelength of 450 nm. These data demonstrate the presence of bacteria-specific bovine antibodies in a preparation of bovine antibodies from non-immunised.

### Example 3

### Allergen-Specific IgG1, IgA or IgM Antibodies in Milk-Derived Antibody Preparations

Sandwich ELISAs to measure grass allergen-specific antibodies are used to quantify the amounts of grass allergen-specific IgG1, IgM, and IgA in a preparation containing bovine antibodies. 96 well ELISA plates are coated with 100 μl of grass pollen allergen extract (a mix of several temperate grasses, ALK-Abello, Horsholm, Denmark) in PBS. The plates are incubated overnight at 4° C. in a humidified environment. The plates are washed three times with 200 ul wash buffer per well (PBS+0.05% Tween-20) and blocked for 1 hour at 37° C. in a humidified environment with 200 μL block buffer per well (=wash buffer+1% gelatin). Following this incubation the plates are washed three times with 200 ul wash buffer per well (PBS+0.05% Tween-20), and 100 μL/well of bovine antibody containing sample diluted in block buffer is added. After a 1 hour incubation at 37° C. in a humidified environment, the plates are washed three times with 200 ul wash buffer per well, and 100 μL anti-bovine IgG1-HRP (AbD Serotec, cat# AAI21P) diluted 1:50.000 in block buffer is added per well to detect grass pollen-specific IgG1. For detection of IgA 100 μL anti-bovine IgA-HRP (AbD Serotec, cat# AAI20P) diluted to 1:50.000 in block buffer, and for detection of bovine IgM 100 μL anti-bovine IgM-HRP diluted to 1:50.000 in block buffer is used (AbD Serotec, cat# AAI19P).

The plates are incubated for 1 hour at 37° C. in a humidified environment, washed three times with 200 ul wash buffer per well, followed by a fourth wash with PBS, and 100 μL TMB substrate is added to each of the wells (100 μL TMB in 10 mL substrate buffer both from Biosource Int. Cat # 45.011.03 and 45.014.01)

The color reaction is stopped by adding 100 μL/well of 1M H₂SO₄, and the plates are read at a wavelength of 450 nm. The presence of grass allergen-specific bovine IgG1, IgA, and IgM is thus demonstrated in a preparation of bovine antibodies from non-immunised cows, suggesting that eating grass induces an allergen-specific antibody response in ruminants. This indicates that feeding potential food allergens to ruminants may be a way to induce (food)allergen-specific antibody responses in milk without the need to vaccinate said ruminants.

### Example 4

Bovine milk-derived IgG1 antibodies specific for grass allergens prevent the binding of IgE-allergen complexes to CD23, the low affinity IgE receptor and prevent basophil histamine release.

When allergen extracts are precomplexed to human IgE antibodies specific for theses allergens these complexes can bind to the low affinity IgE receptor CD23 (expressed on B cells, monocytes and dendritic cells) and to the high affinity IgE receptor present on basophils and mast cells. This in turn leads to highly efficient allergen presentation to Th2 cells leading to a prolonged allergic response as well as to the degranulation of basophils and mast cells. It is possible to prevent this antigen presentation as well as the release of inflammatory mediators from basophils and mastcells by introducing an allergen-specific blocking antibody of a class different from IgE, preferably an IgG antibody.

Grass pollen allergen-IgE complex binding to CD23 is investigated via the following manner. A serial dilution of grass pollen allergen extract (a mix of several temperate grasses, ALK-Abello, Horsholm, Denmark) in the absence or the presence of a bovine antibody preparation containing grass pollen allergen-specific IgG1 antibodies is prepared in a volume of 20 ul of FACS buffer (PBS, 0.1% BSA, 0.05% NaN3) in 4 ml FACS tubes. After a 30 minute incubation at 37 C, 30 uL patient serum containing grass pollen allergen-specific IgE is added and the serum and allergen are allowed to form allergen-IgE complexes for an hour at 37° C. in humidified atmosphere. After this incubation, 50.000 EBV transformed B cells that express high levels of CD23, the low affinity IgE receptor are added and the cells are incubated for one hour at 4° C., allowing the IgE-allergen complexes to bind to CD23. After this incubation period the cells are washed with 4 mL FACS buffer and centrifuged for 5 minutes at 1,500 RPM. After washing, 50 uL of FITC-conjugated antibody to IgE (a goat anti-human polyclonal antibody; Kirkegaard & Perry Laboratories) are added to the cells in the presence of 5% normal goat serum to prevent non-specific binding of the FITC labeled anti-IgE antiserum. The tubes are incubated for half an hour at 4° C., cells were washed with 4 mL FACS buffer and centrifuged for 5 minutes at 1,500 RPM. 200 uL FACS buffer is added per tube and the tubes are measured directly with a FACSCalibur flowcytometer. 10,000 events are recorded.

The binding of grass allergen-specific IgE to CD23 expressed on an EBV-transformed B cell line is thus demonstrated in the presence of limited amounts of grass allergen. This binding is reduced by adding a bovine milk-derived preparation containing grass pollen allergen-specific IgG1 antibodies. Similarly it is demonstrated in a histamine release experiment from human peripheral blood basophils from grass pollen allergic patients that histamine release by the basophils is inhibited in the presence of bovine milk-derived preparation containing grass pollen allergen-specific IgG1 antibodies. In this experiment, human peripheral blood mononuclear cells (PBMC) are isolated from heparinized blood from a grass pollen-allergic patient by density centrifugation. The PBMC are collected and incubated with a serial dilution of grass pollen allergens that has been preincubated for 30 minutes at 37 C with or without a bovine milk-derived preparation containing grass pollen allergen-specific IgG1 antibodies. The PBMC are incubated for 30 minutes at 37 C, after which the supernatant is removed to measure the amount of histamine released using a commercial histamine determination kit. In this manner it is demonstrated that the release of histamine by human basophils is reduced by the presence of bovine grass pollen allergen-specific IgG1 antibodies.

### Example 5

Transport of bovine IgG1 (and IgM, IgA) over monolayers of the human intestinal epithelium cell line Caco-2. The human small intestinal epithelial cell line Caco-2 is cultivated in Transwell plates and allowed to differentiate into monolayers during a 15-21 day culture period. Monolayer integrity is measured every week by measuring the transepithelial electrical resistance (TEER) before use in transport assays. To measure transport of bovine antibodies, a preparation containing bovine antibodies (see example 1) is added to the Caco-2 monolayers and samples are taken at several time points to measure the transport of bovine antibodies. This experiment is performed in the absence or presence of an inducer of paracellular transport (sodium caprate) or an inhibitors of transcellular transport (an 8 hr pretreatment of the cells with actinomycin D). In addition, a FITC-labeled dextran is used as an additional control in all wells to visualize paracellular transport of small molecules. The antibody levels transported across the Caco-2 monolayer is measured by a sandwich ELISA developed for the measurement of total IgG1, IgM, and IgA antibodies similar to the ELISAs described in previous examples. In short, 96 well ELISA plates are coated with 100 μL sheep anti-bovine IgG diluted 1:800 in PBS (as IgG capture antibody—Bethyl Cat # A10-118A-8) in PBS, 100 μL sheep anti bovine IgA diluted in PBS per well (IgA capture antibody—Bethyl cat #A10-121A-8), or 100 μl sheep anti bovine IgM diluted in PBS per well (IgM capture antibody—Bethyl cat #A10-101A-8). The plates are incubated overnight at 4° C. in a humidified environment. Following this incubation the plates are washed three times with 200 ul wash buffer per well (PBS+0.05% Tween-20) and blocked for 1 hour at 37° C. in a humidified environment with 200 μL block buffer per well (=wash buffer+1% gelatin). The plates are washed three times with 200 ul wash buffer per well (PBS+0.05% Tween-20), and 100 μL/well of bovine antibody containing sample diluted in block buffer is added.

After a 1 hour incubation at 37° C. in a humidified environment, the plates are washed three times with 200 ul wash buffer per well, and 100 μL anti-bovine IgG1-HRP (AbD Serotec, cat# AAI21P) diluted 1:50.000 in block buffer is added per well to detect the presence of bovine IgG1. For detection of IgA 100 μL anti-bovine IgA-HRP (AbD Serotec, cat# AAI20P) diluted to 1:50.000 in block buffer, and for detection of bovine IgM 100 μL anti-bovine IgM-HRP (AbD Serotec, cat# AAI19P) diluted to 1:50.000 in block buffer is used. The plates are incubated for 1 hour at 37° C. in a humidified environment, washed three times with 200 ul wash buffer per well, followed by a fourth wash with PBS, and 100 μL TMB substrate is added to each of the wells (100 μL TMB in 10 mL substrate buffer both from Biosource Int. Cat # 45.011.03 and 45.014.01). The total amounts of bovine antibodies present in the samples are quantified against a standard curve of a standard bovine serum containing bovine IgG1, IgA, and IgM (Bethyl Cat # RS10-103).

The transport of bovine antibodies over the Caco-2 monolayer is thus demonstrated. In addition, reduction of the transport by pretreatment of the cells with actinomycin-D, and enhancement of transport by the addition of sodium caprate, an inducer of paracellular transport, indicates that bovine antibodies are actively transported over human intestinal epithelia.

### Example 6

### Food Allergen-Specific Bovine IgG Antibodies can Prevent Allergic Reactions Upon Ingestion of Food Allergens

Cows are immunized with a peanut protein extract until sufficiently high peanut-specific antibody levels are detected in milk from the cows. Milk is collected, antibodies are isolated and purified either from whey after cheese making or directly from the milk—if possible without heating the milk. Peanut allergic patients are selected and divided into two groups. Both groups undergo a double blind, placebo controlled food challenge to determine a threshold level for peanut sensitivity. This threshold is defined as the amount of peanut ingested that induces allergic symptoms in the patient. Following this challenge one group is placed on a diet containing an antibody preparation from peanut-immunized cows, and the control group is given a diet containing a control bovine antibody preparation (non-immunized cows). After a period of three weeks to three months on the diet all patients undergo a second double blind, placebo controlled food challenge to determine their peanut threshold. At both timepoints blood samples are collected to determine the amount of bovine antibodies (both peanut-specific and total bovine antibody levels). These antibody levels are detected using the ELISAs described in example 6 (for total antibody levels) and the ELISAs described in example 3, with the modification that instead of grass pollen allergen extract a peanut allergen extract is coated onto the surface of the ELISA plates.

The results of this study indicate that the ingestion of peanut allergen-specific bovine antibodies increase the amount of peanut allergen a peanut allergic patient can tolerate, and suggest that oral delivery of bovine allergen-specific antibodies may be a good method to reduce allergic reactions in allergic patients. In addition, the study demonstrates that antigen-specific bovine antibodies (peanut-specific antibodies in this case) are taken up into the blood of food-allergic patients, suggesting that it is possible to systemically deliver a bovine, antigen-specific antibody to a human individual.

### Example 7

### Pathogen-Specific Antibody Levels Present in Milk-Derived Antibody Preparations

ELISAs were used to detect the presence of bacteria-specific IgG1 and IgA in colostrum (dissolved at 20 mg/ml), milk, and whey, all from non-immunized cows. 96 well ELISA plates were coated by adding to each well 100 μl of 10 μg/ml of commercially available LPS from E. Coli or Salmonella typhimurium (Sigma), diluted in PBS. The plates were incubated overnight at 4° C. in a humidified environment. The plates were washed three times with 200 ul wash buffer per well (PBS+0.05% Tween-20) and blocked for 1 hour at 37° C. in a humidified environment with 200 μL block buffer per well (=wash buffer+1% gelatin). After this incubation, the plates were washed three times with 200 ul wash buffer per well (PBS+0.05% Tween-20), and 100 μL/well of serially diluted bovine immunoglobulin containing sample diluted in block buffer were added. After a 1 hour incubation at 37° C. in a humidified environment, the plates were washed three times with 200 ul wash buffer per well, and 100 μL anti-bovine IgG1-HRP (AbD Serotec, cat# AAI21P) diluted 1:20.000 in block buffer was added per well to detect bacteria-specific IgG1. For detection of IgA 100 μL anti-bovine IgA-HRP (AbD Serotec, cat# AAI20P) diluted to 1:50.000 in block buffer was used.

The plates were incubated for 1 hour at 37° C. in a humidified environment, washed three times with 200 ul wash buffer per well, followed by a fourth wash with PBS, and 100 μL TMB substrate was added to each of the wells (100 μL TMB in 10 mL substrate buffer both from Biosource Int. Cat # 45.011.03 and 45.014.01).

The color reaction was stopped by the addition of 100 μL/well of 1M H2SO4, and the plates were read at a wavelength of 450 nm.

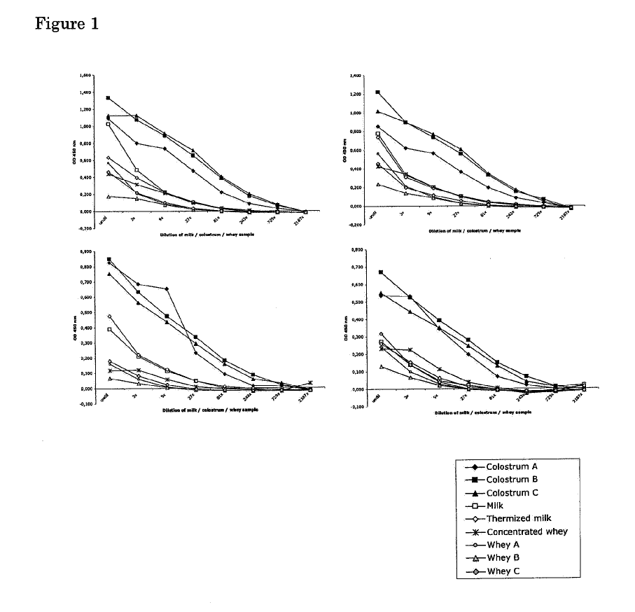

As shown in FIG. 1, colostrum contains IgA and IgG1 immunoglobulins that react specifically with LPS from Escherichia coli and Salmonella typhimurium. These LPS-specific immunoglobulins are also detected in milk and whey, though at a lower concentration. These data demonstrate the presence of bacteria-specific bovine antibodies in colostrum, milk and whey from non-immunised cows.

### Example 8

### Allergen-Specific Antibody Levels Present in Milk-Derived Antibody Preparations

ELISAs were used to determine the presence of allergen-specific IgG1, and IgA in colostrum from non-immunized cows. 96 well ELISA plates were coated with serial dilutions of allergenic protein extracts from house dust mite Dermatophagoides pteronyssinus (Dp), birch pollen (Betula verrucosa), or grass pollen (all from ALK Abello, Horsholm, Denmark) diluted in PBS. The plates were incubated overnight at 4° C. in a humidified environment. The plates were washed three times with 200 ul wash buffer per well (PBS+0.05% Tween-20) and blocked for 1 hour at 37° C. in a humidified environment with 200 μL block buffer per well (=wash buffer+1% gelatin). After this incubation, the plates were washed three times with 200 ul wash buffer per well (PBS+0.05% Tween-20), and 100 μL/well of bovine immunoglobulin containing sample diluted in block buffer is added. After a 1 hour incubation at 37° C. in a humidified environment, the plates were washed three times with 200 ul wash buffer per well, and 100 μL anti-bovine IgG1-HRP (AbD Serotec, cat# AAI21P) diluted 1:20.000 in block buffer was added per well to detect bacteria-specific IgG1. For detection of IgA 100 μL anti-bovine IgA-HRP (AbD Serotec, cat# AAI20P) diluted to 1:50.000 in block buffer was used.

The plates were incubated for 1 hour at 37° C. in a humidified environment, washed three times with 200 ul wash buffer per well, followed by a fourth wash with PBS, and 100 μL TMB substrate was added to each of the wells (100 TMB in 10 mL substrate buffer both from Biosource Int. Cat # 45.011.03 and 45.014.01).

The color reaction was stopped by the addition of 100 μL/well of 1M H2SO4, and the plates were read at a wavelength of 450 nm.

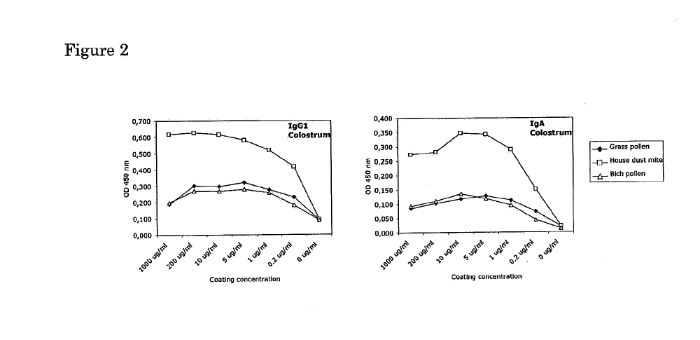

As shown in FIG. 2, colostrum contains IgA as well as IgG1 immunoglobulins that react specifically with housedust mite-, birch pollen-, and grass pollen-allergens. These data demonstrate the presence of allergen-specific bovine antibodies in colostrum from non-immunised cows.

### Example 9

### Transport of Bovine IgG1 and IgA Over Monolayers of the Human Intestinal Epithelium Cell Line Caco-2

The human small intestinal epithelial cell line Caco-2 was cultivated in Transwell plates and allowed to differentiate into monolayers during a 15-21 day culture period. Monolayer integrity was measured every week by measuring the transepithelial electrical resistance (TEER) before use in transport assays. Experiments were performed after the TEER has reached a value of at least 500 ohm/cm2. To measure transport of bovine immunoglobulins, bovine colostrum containing bovine immunoglobulins was diluted to a concentration of 20 mg/ml (w/v corresponding to 2.4 mg/ml IgG1 and 0.15 mg/ml IgA) in a 10 mM MES buffer with pH 6.0, 6.5 and pH 7 and added to the apical side of the Caco-2 monolayers. The pH at the basolateral side of the Caco-2 cells was 7.4, thus creating a pH gradient as occurs in the human intestine. In addition, FITC-labeled dextran (FD4, 0.01 mg/ml) was added to the medium at the apical side as a control in all wells to visualize paracellular transport of small molecules to exclude that transport measured is the result of a leaky monolayer. TEER was measured again after a 4 hour incubation period, and samples were taken at the basolateral side to measure the transport of bovine immunoglobulins as well as leakage of FD4. The immunoglobulin levels transported across the Caco-2 monolayer were measured by a sandwich ELISA developed for the measurement of total bovine IgG1 and IgA. In short, 96 well ELISA plates were coated with 100 μL sheep anti-bovine IgG diluted 1:800 in PBS (as IgG capture immunoglobulin Bethyl Cat # A10-118A-8) in PBS, 100 μL sheep anti bovine IgA diluted in PBS per well (IgA capture immunoglobulin—Bethyl cat #A10-121A-8), or 100 μL sheep anti bovine IgM diluted in PBS per well (IgM capture immunoglobulin—Bethyl cat #A10-101A-8). The plates were incubated overnight at 4° C. in a humidified environment. Following this incubation the plates were washed three times with 200 ul wash buffer per well (PBS+0.05% Tween-20) and blocked for 1 hour at 37° C. in a humidified environment with 200 μL block buffer per well (=wash buffer+1% gelatin; DIFCO cat #214340). The plates were washed three times with 200 ul wash buffer per well (PBS+0.05% Tween-20), and 100 μL/well of bovine immunoglobulin containing sample diluted in block buffer was added.

After a 1 hour incubation at 37° C. in a humidified environment, the plates were washed three times with 200 ul wash buffer per well, and 100 μL anti-bovine IgG1-HRP (AbD Serotec, cat# AAI21P) diluted 1:20.000 in block buffer was added per well to detect the presence of bovine IgG1. For detection of IgA 100 μL anti-bovine IgA-HRP (AbD Serotec, cat# AAI20P) diluted to 1:50.000 in block buffer, and for detection of bovine IgM 100 μL anti-bovine IgM-HRP (AbD Serotec, cat# AAI19P) diluted to 1:50.000 in block buffer was used. The plates were incubated for 1 hour at 37° C. in a humidified environment, washed three times with 200 ul wash buffer per well, followed by a fourth wash with PBS, and 100 μL TMB substrate was added to each of the wells (100 μL TMB in 10 mL substrate buffer both from Biosource Int. Cat # 45.011.03 and 45.014.01). The color reaction was stopped by the addition of 100 μL/well of 1M H2SO4, and the plates were read at a wavelength of 450 nm. The total amounts of bovine IgG1, IgA and IgM immunoglobulins present in the samples were quantified against a standard curve of a standard bovine serum containing bovine IgG1, IgA, and IgM Methyl Cat # RS10-103).

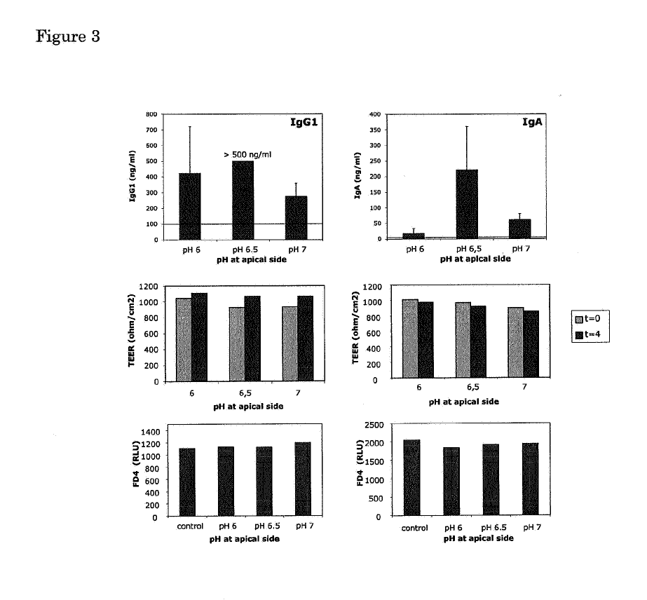

FIG. 3 shows that both IgA and IgG1 are transported from the apical to the basolateral side, especially at a pH of 6.5. The amount of IgG1 and IgA transported is relatively low, but no leakage in the epithelium was induced by the addition of colostrum as detected by measuring FITC-labeled dextran transported paracellularly to the basolateral side, as well as by measuring TEER, indicating bovine IgA and IgG1 can be actively transported over human intestinal epithelia.

1-54. (canceled)

55. A method for obtaining an antigen-specific antibody from milk, which method comprises providing milk derived from at least ten individual ruminants of the same species that have not been immunized with said antigen prior to collecting said milk, pooling the milk prior to and collecting said antibody from the pooled milk.

56. A method according to claim 55, further comprising a step to specifically collect IgG₁, IgA or a combination thereof.

57. A method according claim 55, wherein said ruminants are selected for collection of said milk based on the antibody content of the milk, antibody specificity in collected milk, food or food-supplement ingestion by said ruminants, vaccination for an antigen of a pathogen of said ruminants, or a combination thereof.

58. A method according to claim 55, wherein said species is cow, goat or sheep.

59. A method according to claim 55, wherein said antigen is an allergy antigen, a viral antigen, a bacterial antigen or a combination thereof.

60. A method according to claim 55, wherein said antigen is birch pollen.

61. A method according claim 55, which comprises preparing a whey fraction from said milk and collecting said antibody from said whey.

62. A method according to claim 55, further comprising performing an affinity purification step to obtain an antibody fraction that is enriched for said antigen-specific antibody.

63. An antigen-specific polyclonal antibody obtainable by a method according to claim 55.

64. A composition comprising an antibody according to claim 63, wherein said antigen-specific antibody comprises less than 0.1% of the total amount of antibody in said composition.

65. A newborn or infant formula comprising an antibody according to claim 63.

66. A method for the systemic treatment of a disease in a human, comprising orally administrating the antigen-specific antibody of claim 63.

67. A composition according to claim 64, further comprising a prebiotic.

68. A composition comprising an antigen-specific antibody obtainable by a method according to claim 56, wherein at least 15% of the antibodies are IgG₁, IgA or a combination thereof.

69. A method for enhancing transport of an antigen-specific antibody from the apical side to the basolateral side of intestinal epithelial cells, comprising providing a composition of claim 64, which composition further comprises a prebiotic.

70. A method for delivering an antigen-specific antibody of a ruminant to the mucosal side of the intestinal tract of an individual, comprising providing a preparation for oral administration comprising said antibody of a ruminant and orally administering said preparation to said individual.
